(19) United States
(12) Patent Application Publication
(10) Pub. No.: US 2026/0325210 A1
(43) Pub. Date: Oct. 22, 2026

(54) LASER LIGHT IRRADIATION SYSTEM, LASER CONTROL DEVICE, AND LASER LIGHT IRRADIATION METHOD

(71) Applicant: OLYMPUS CORPORATION, Tokyo (JP)

(72) Inventor: Takumi HAYASHI, Tokyo (JP)

(73) Assignee: OLYMPUS CORPORATION, Tokyo (JP)

(21) Appl. No.: 19/634,087

(22) Filed: Mar. 31, 2026

Related U.S. Application Data
(63) Continuation of application No. PCT/JP2023/043062, filed on Dec. 1, 2023.

(51) Int. Cl. A61B 18/26; A61B 18/00

(52) U.S. Cl. CPC A61B 18/26; A61B 2018/00732; A61B 2018/00982

(57) ABSTRACT

A laser light irradiation system (1) for irradiating a calculus (S) present in the body with laser light to fragment the calculus (S) in a liquid comprises: a laser fiber (3) having a fiber emission end (3a) from which the laser light is emitted; and at least one processor that controls the frequency of the laser light emitted from the fiber emission end (3a). The processor is configured to switch the frequency of the laser light between a first pulse group having a high frequency and a power output for fragmenting the calculus (S), and a second pulse group having a frequency lower than that of the first pulse group and a waveform in which the peak power gradually increases in the direction of the axis for irradiation time, and emit the second pulse group at least either before or after the timing of emitting the first pulse group.

### CROSS-REFERENCE TO RELATED APPLICATION

This application is a continuation of PCT International Application No. PCT/JP2023/043062, filed on Dec. 1, 2023. The above application is hereby expressly incorporated by reference, in its entirety, into the present application.

### BACKGROUND

### Technical Field

The disclosure relates to a laser light irradiation system, a laser control device, and a laser light irradiation method.

### Description of Related Art

Conventionally, a technique for fragmenting calculi in kidneys using laser light is known (for example, see Patent Document 1). A calculus is pulverized to a desired size by laser irradiation, from a stage where the calculus of a larger size is fragmented (dusting) through a stage where small calculus fragments created by fragmentation flutter around (popcorn dusting). The technique described in Patent Document 1 varies the frequency of the laser light as one variable for pulverizing calculi by laser irradiation.

### PRIOR ART DOCUMENT(S)

### Patent Document(s)

Patent Document 1: International Patent Publication No. WO 2020/033121

The disclosure provides a laser light irradiation system, a laser control device, and a laser light irradiation method that can reduce wasteful firing of laser light and shorten surgery time.

### SUMMARY

According to a first aspect of the disclosure, a laser light irradiation system that fragments a calculus in liquid by irradiating laser light to the calculus present in a body. The laser light irradiation system includes: a laser fiber, having a fiber emission end that emits the laser light; and at least one processor, configured to control a frequency of the laser light emitted from the fiber emission end. The processor is configured to switch the frequency of the laser light between a high-frequency first pulse group having an output that fragments the calculus and a second pulse group having a frequency lower than the first pulse group and having a waveform in which a peak power gradually increases in an axial direction of irradiation time, and emit the second pulse group at least one of before and after a timing of emitting the first pulse group.

A second aspect of the disclosure provides a laser control device. The laser control device includes: at least one processor, configured to control a frequency of laser light emitted from a fiber emission end of a laser fiber toward a calculus present in a body. The processor is configured to switch a frequency of the laser light between a high-frequency first pulse group having an output that fragments the calculus and a second pulse group having a frequency lower than the first pulse group and having a waveform in which a peak power gradually increases in an axial direction of irradiation time, and emit the second pulse group at least one of before and after a timing of emitting the first pulse group.

A third aspect of the disclosure provides a laser light irradiation method for fragmenting a calculus in liquid by irradiating laser light to the calculus present in a body. The laser light irradiation method includes: emitting a high-frequency first pulse group having an output for fragmenting the calculus from a fiber emission end toward the calculus; and at least one of before and after a timing of emitting the first pulse group, emitting a second pulse group having a frequency lower than the first pulse group and having a waveform in which a peak power gradually increases in an axial direction of irradiation time.

### BRIEF DESCRIPTION OF THE DRAWINGS

FIG. 1 is a schematic diagram showing a configuration of a laser light irradiation system according to an embodiment of the disclosure.

FIG. 2 is a view showing a state in which a laser fiber is inserted into a working channel of an endoscopic scope.

FIG. 3 is a view showing control conditions for the frequency of laser light being determined by a determination part from information acquired by a sensor.

FIG. 4 is a table showing an example of frequency of laser light, etc., in a dusting mode and a popcorning mode.

FIG. 5 is a view showing selecting a mode stored in a storage part by using a touch panel.

FIG. 6 is a diagram showing a user manually inputting control conditions by using a touch panel.

FIG. 7 is a block diagram showing a configuration of a laser control device.

FIG. 8 is a diagram showing an example of a switching pattern between a first pulse group and a second pulse group.

FIG. 9 is a diagram showing a pulse width of a first pulse group.

FIG. 10 is a diagram showing a duty ratio of the first pulse group.

FIG. 11 is a diagram showing a second pulse group of a triangular waveform.

FIG. 12 is a flowchart showing a laser light irradiation method according to an embodiment of the disclosure.

FIG. 13 is a diagram showing a pattern of emitting the first pulse group by switching the frequency of the laser light after firstly emitting the second pulse group.

FIG. 14 is a diagram showing a pattern of emitting the second pulse group both before and after emission of the first pulse group.

FIG. 15 is a diagram showing a first pulse group having a waveform that gradually increases in a stepwise manner in the axial direction of irradiation time.

FIG. 16 is a diagram showing a second pulse group of an M-shaped waveform.

FIG. 17 is a diagram showing a second pulse group of a U-shaped waveform.

FIG. 18 is a diagram showing a second pulse group of a rectangular wave waveform.

FIG. 19 is a diagram showing an example of a pattern in which the pulse count of the first pulse group differs or the waveform of the second pulse group differs per pulse set.

FIG. 20 is a diagram showing a movement in which a calculus irradiated with laser light scatters in a renal calyx where the approach direction faces the horizontal direction.

FIG. 21 is a diagram showing a movement in which a calculus irradiated with laser light scatters in a renal calyx where the approach direction faces the gravity direction.

### DESCRIPTION OF THE EMBODIMENTS

According to a first aspect of the disclosure, a laser light irradiation system that fragments a calculus in liquid by irradiating laser light to the calculus present in a body. The laser light irradiation system includes: a laser fiber, having a fiber emission end that emits the laser light; and at least one processor, controlling a frequency of the laser light emitted from the fiber emission end. The processor switches the frequency of the laser light between a high-frequency first pulse group having an output that fragments the calculus and a second pulse group having a frequency lower than the first pulse group and having a waveform in which a peak power gradually increases in an axial direction of irradiation time, and emits the second pulse group at least one of before and after a timing of emitting the first pulse group.

According to this aspect, the laser light of the first pulse group has high fragmentation capability but tends to scatter the calculus. On the other hand, the laser light of the second pulse group has low fragmentation capability but has a high suction effect that attracts the calculus to the fiber emission end within a certain range from the fiber emission end. In particular, the laser light of the second pulse group has a higher suction effect than the case of having a rectangular wave waveform by having a waveform in which the peak power gradually increases in the axial direction of irradiation time.

Therefore, with the processor emitting the laser light of the first pulse group and the laser light of the second pulse group at different timings, the fragmentation effect exerted by the laser light of the first pulse group and the suction effect exerted by the laser light of the second pulse group can compensate for the respective disadvantages, and the fragmentation efficiency can be improved. Accordingly, suppress wasteful firing of laser light in which laser light is emitted in a state where the calculus is positioned at a location separated from the fiber emission end can be suppressed, and the surgery time can be reduced.

In the laser light irradiation system according to the above aspect, it may also be that a waveform of the first pulse group is a multi-pulse train.

With the configuration, the laser light of the first pulse group can be intensively irradiated to the calculus in a short time before the calculus scatters.

In the laser light irradiation system according to the above aspect, it may also be that the waveform of the second pulse group is a triangular waveform in which a peak power gradually increases in the axial direction, or an M-shaped waveform in which a peak power gradually decreases and then gradually increases in the axial direction.

With this configuration, in the case where the second pulse group has a triangular waveform, after multiple bubbles are continuously generated from the fiber tip, adjacent bubbles are combined with each other to form a snowman-shaped clustered bubbles. Accordingly, the suction effect be enhanced. On the other hand, in the case where the second pulse group has an M-shaped waveform, the laser light with a high peak power is emitted temporally separated with laser light with a low peak power in between. Accordingly, two bubbles are generated separately from the fiber tip. Thus, the suction effect can be enhanced.

In the laser light irradiation system according to the above aspect, it may also be that the processor performs a pulse set multiple times. The pulse set provides a stop period for stopping emission of the laser light for a predetermined period after an emission period including a period of emission of the first pulse group and a period of emission of the second pulse group.

With this configuration, since the laser light is not emitted during the stop period, the average power of the laser light emitted in each pulse set can be suppressed. Accordingly, an increase in the patient's intrarenal temperature can be suppressed.

In the laser light irradiation system according to the above aspect, it may also be that the processor determines a power of the first pulse group, a power of the second pulse group, and a length of the stop period within a range where an average power of the laser light emitted in the pulse set is 20 W or less.

In the laser light irradiation system according to the above aspect, it may also be that the average power is calculated by Equations (1), (2), and (3) as follows:

[Equation 1]

PE=PE_{A}·N_{A}+PE_{H}·N_{H} (1)

$$[ Equation 2 ]$$

$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} , and \qquad ( 2 )$$

$$[ Equation 3 ]$$

$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} , \qquad ( 3 )$$

where Pₐᵥₑ represents the average power, PE_{A} and PE_{H} represent pulse energies, f_{A} and f_{H} represent frequencies, N_{A} and N_{H} represent pulse counts, and Tᵢₙₜ represents the stop period.

In the laser light irradiation system according to the above aspect, it may also be that the processor performs a second pulse set in which, following the first pulse set, an emission period is followed by the stop period. The emission period includes: a period of emission of a third pulse group having an output for fragmenting the calculus and having a high frequency different from the first pulse group; and a period of emission of a fourth pulse group having a frequency different from the second pulse group and lower than the third pulse group and having a waveform in which a peak power gradually increases in the axial direction of irradiation time.

The laser light irradiation system according to the above aspect may further include: an endoscope, able to be inserted into the body. The endoscope includes a channel that allows the fiber emission end to protrude forward in a state where the laser fiber penetrates.

A second aspect of the disclosure provides a laser control device. The laser control device includes: at least one processor, controlling a frequency of laser light emitted from a fiber emission end of a laser fiber toward a calculus present in a body. The processor switches a frequency of the laser light between a high-frequency first pulse group having an output that fragments the calculus and a second pulse group having a frequency lower than the first pulse group and having a waveform in which a peak power gradually increases in an axial direction of irradiation time, and emits the second pulse group at least one of before and after a timing of emitting the first pulse group.

In the laser control device according to the above aspect, it may also be that a waveform of the first pulse group is a multi-pulse train.

In the laser control device according to the above aspect, it may also be that the waveform of the second pulse group is a triangular waveform in which a peak power gradually increases in the axial direction, or an M-shaped waveform in which a peak power gradually decreases and then gradually increases in the axial direction.

In the laser control device according to the above aspect, the processor performs a pulse set multiple times. The pulse set provides a stop period for stopping emission of the laser light for a predetermined period after an emission period including a period of emission of the first pulse group and a period of emission of the second pulse group.

In the laser control device according to the above aspect, it may also be that the processor determines a power of the first pulse group, a power of the second pulse group, and a length of the stop period within a range where an average power of the laser light emitted in the pulse set is 20 W or less.

In the laser control device according to the above aspect, it may also be that the average power is calculated by Equations (1), (2), and (3) as follows:

[Equation 1]

PE=PE_{A}·N_{A}+PE_{H}·N_{H} (1)

$$[ Equation 2 ]$$

$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} , and \qquad ( 2 )$$

$$[ Equation 3 ]$$

$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} , \qquad ( 3 )$$

where Pₐᵥₑ represents the average power, PE_{A} and PE_{H} represent pulse energies, f_{A} and f_{H} represent frequencies, N_{A} and N_{H} represent pulse counts, and Tᵢₙₜ represents the stop period.

In the laser control device according to the above aspect, it may also be that the processor performs a second pulse set in which, following the first pulse set, an emission period is followed by the stop period. The emission period includes: a period of emission of a third pulse group having an output for fragmenting the calculus and having a high frequency different from the first pulse group; and a period of emission of a fourth pulse group having a frequency different from the second pulse group and lower than the third pulse group and having a waveform in which a peak power gradually increases in the axial direction of irradiation time.

A third aspect of the disclosure provides a laser light irradiation method for fragmenting a calculus in liquid by irradiating laser light to the calculus present in a body. The laser light irradiation method includes: emitting a high-frequency first pulse group having an output for fragmenting the calculus from a fiber emission end toward the calculus; and at least one of before and after a timing of emitting the first pulse group, emitting a second pulse group having a frequency lower than the first pulse group and having a waveform in which a peak power gradually increases in an axial direction of irradiation time.

In the laser light irradiation method according to the above aspect, it may also be that a pulse set is performed multiple times. The pulse set provides a stop period for stopping emission of the laser light for a predetermined period after an emission period including a period of emission of the first pulse group and a period of emission of the second pulse group.

In the laser light irradiation method according to the above aspect, it may also be that a power of the first pulse group, a power of the second pulse group, and a length of the stop period are determined within a range where an average power of the laser light emitted in the pulse set is 20 W or less.

In the laser light irradiation method according to the above aspect, it may also be that the average power is calculated by Equations (1), (2), and (3) as follows:

[Equation 1]

PE=PE_{A}·N_{A}+PE_{H}·N_{H} (1)

$$[ Equation 2 ]$$

$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} , and \qquad ( 2 )$$

$$[ Equation 3 ]$$

$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} , \qquad ( 3 )$$

where Pₐᵥₑ represents the average power, PE_{A} and PE_{H} represent pulse energies, f_{A} and f_{H} represent frequencies, N_{A} and N_{H} represent pulse counts, and Tᵢₙₜ represents the stop period.

In the laser light irradiation method according to the above aspect, it may also be that a second pulse set is performed. In the second pulse set, following the first pulse set, an emission period is followed by the stop period. The emission period includes: a period of emission of a third pulse group having an output for fragmenting the calculus and having a high frequency different from the first pulse group; and a period of emission of a fourth pulse group having a frequency different from the second pulse group and lower than the third pulse group and having a waveform in which a peak power gradually increases in the axial direction of irradiation time.

According to the disclosure, wasteful firing of laser light can be reduced and surgery time can be shortened.

A laser light irradiation system, a laser control device, and a laser light irradiation method according to an embodiment of the disclosure will be described below with reference to the drawings.

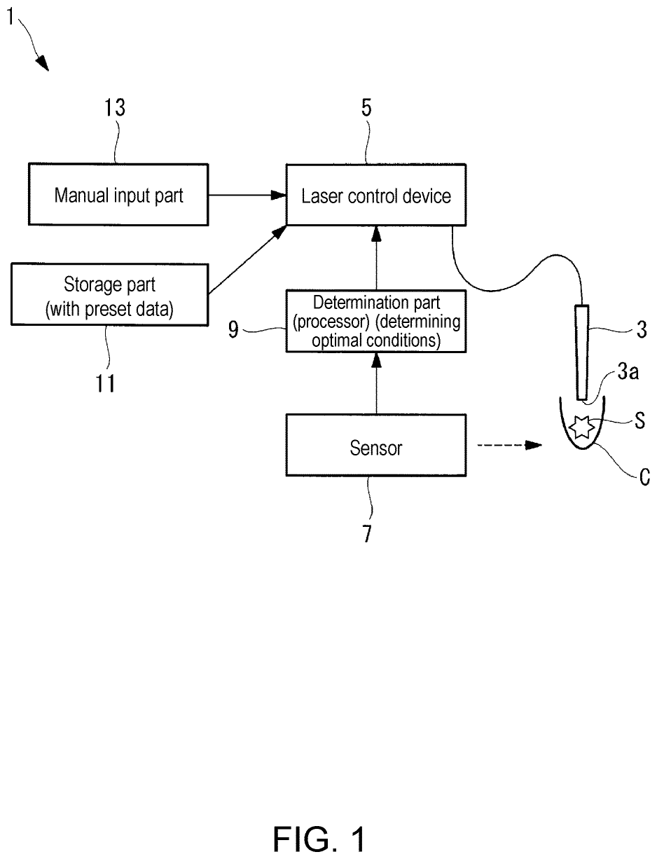

A laser light irradiation system 1 according to the present embodiment, as shown in FIG. 1, fragments a calculus S in liquid by irradiating laser light to the calculus S present in the body. In the figure, a reference sign C indicates a renal calyx where the calculus S remains.

The laser light irradiation system 1 includes: a laser fiber 3 having a fiber emission end 3a that emits the laser light; a laser control device 5 that controls the frequency of laser light emitted from the fiber emission end 3a; a sensor 7 that detects the state of the calculus S; a determination part (processor) 9 that determines control conditions for the frequency of the laser light; a storage part 11 that stores preset data; and a manual input part 13, such as a mouse, a keyboard, and a touch panel. Here, frequency indicates the repetition frequency of pulses.

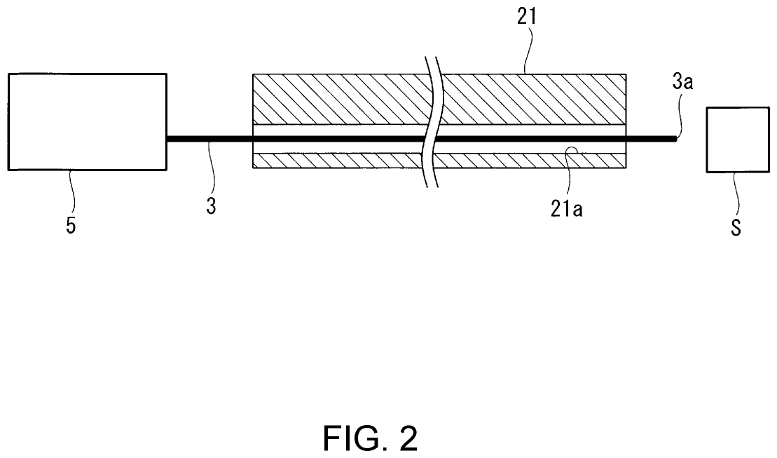

Also, the laser light irradiation system 1 includes an endoscopic scope (endoscope) 21 that can be inserted into the body, as shown in FIG. 2. The endoscopic scope 21 is provided with a working channel (channel) 21a through which the fiber emission end 3a is able to protrude forward in a state where the laser fiber 3 penetrates.

The laser fiber 3 may be, for example, either a single-mode fiber or a multi-mode fiber, and may also be a fiber with a double-clad structure.

The sensor 7 is, for example, a camera. The sensor 7 acquires, for example, an endoscopic image, an X-ray image, or an ultrasound image, etc., of the calculus S in a liquid. Also, the sensor 7 acquires the information relating to the state of the calculus S such as size, number, type, or shape, based on the acquired image of the calculus S, as well as the information regarding the size, shape, or gravity direction of an enclosed space where the laser light is irradiated. Regarding the type of the calculus S, the composition can be examined through laser-induced breakdown spectroscopy or similar methods. The size, number, and shape of the calculus S can be examined by using, for example, an endoscopic image, the X-ray fluoroscopy, or an ultrasound image, etc.

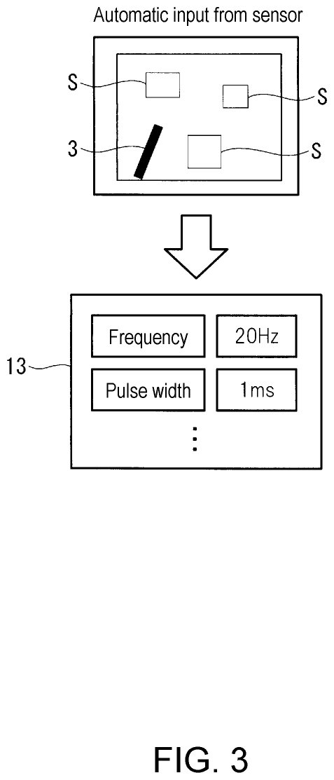

As shown in FIG. 3, the determination part 9 determines optimal control conditions for controlling the frequency of the laser light based on the information acquired by the sensor 7, namely the information regarding the state of the calculus S and the information regarding the state of the space. Since the manner of breaking the calculus S differs according to the size, number, type, or shape of the calculus S, or the size, shape, or gravity direction of the enclosed space where the laser light is irradiated, such configuration allows the calculus S to be fragmented more efficiently. The processing performed by the determination part 9 is executed by at least one processor including hardware. The determination of each parameter in determining the optimal control conditions by the determination part 9 will be described later.

The storage part 11 stores preset data of multiple control conditions for controlling the frequency of the laser light. The preset data may be, for example, a compilation of optimal laser light frequencies obtained by conducting experiments in advance while changing the frequency of the laser light according to the size, number, type, or shape of the calculus S, or the size, shape, or gravity direction of the enclosed space where the laser light is irradiated.

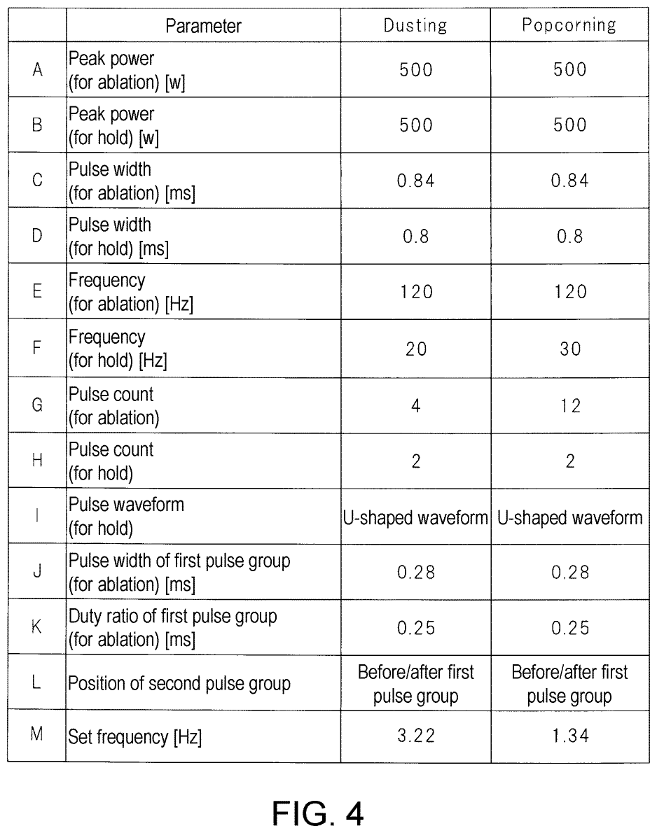

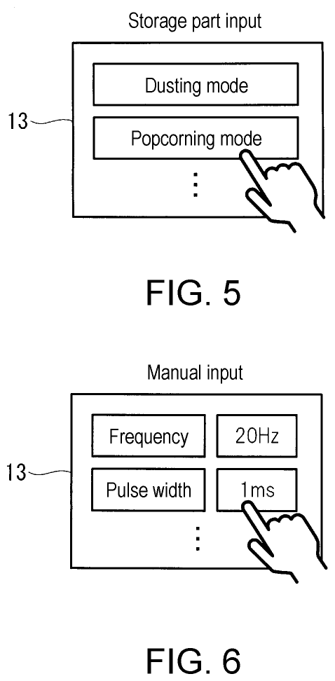

For example, as shown in FIG. 4, the optimal frequencies of the laser light may be compiled according to the stages of fragmentation, such as a dusting mode for the stage of fragmenting a large calculus and a popcorning mode for the stage of further fragmenting the calculus that has become smaller in size. In this case, for example, as shown in FIG. 5, the optimal control conditions according to the stages of fragmentation can be set simply by the user switching modes by using the manual input part 13 or a foot switch (not shown).

The manual input part 13 allows the user to input the desired control conditions for controlling the frequency of the laser light. For example, as shown in FIG. 6, the user may manually input the desired control conditions by using the manual input part 13.

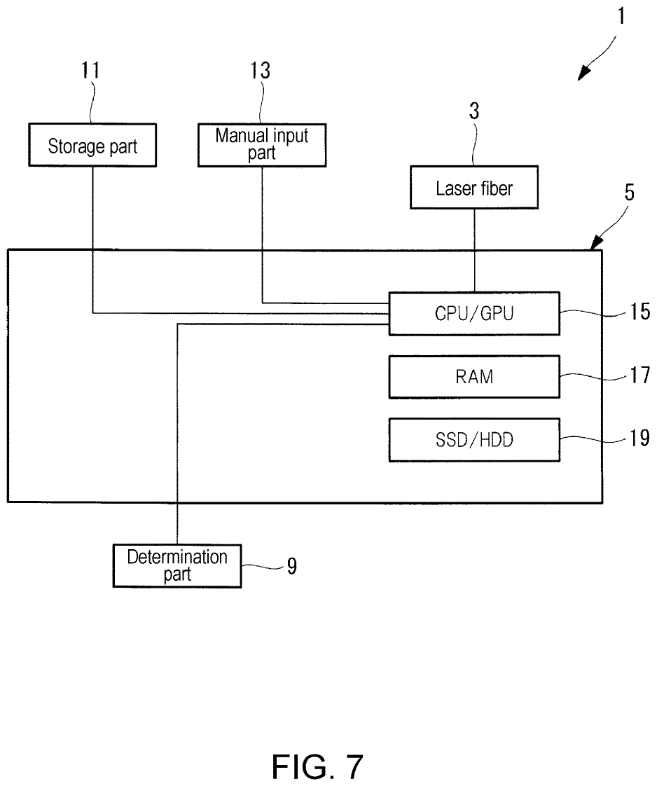

The laser control device 5 is realized by, for example, a dedicated or general-purpose computer. That is, the laser control device 5 includes, as shown in FIG. 7, at least one processor 15 such as a central processing unit (CPU) or a graphics processing unit (GPU), a main storage device 17, and an auxiliary storage device 19.

The main storage device 17 is a random access memory (RAM) or the like used as a working area of the processor 15.

The auxiliary storage device 19 is a non-transitory computer-readable recording medium such as a solid state drive (SSD) or a hard disk drive (HDD). The auxiliary storage device 19 stores a control program that causes the processor 15 to execute processing. The main storage device 17 and the auxiliary storage device 19 may be connected to the laser control device 5 via a network.

Additionally, the laser control device 5 is equipped with a light source unit (not shown) that oscillates the laser light for fragmenting the calculus S. The laser light oscillated from the light source unit is guided by the laser fiber 3 and then emitted from the fiber emission end 3a.

The laser control device 5 receives an input of any one of the control conditions input by the manual input part 13, the control conditions stored in the storage part 11, and the control conditions determined by the determination part 9, according to user settings. The laser control device 5 then switches the frequency of the laser light oscillated from the light source unit based on the input control conditions.

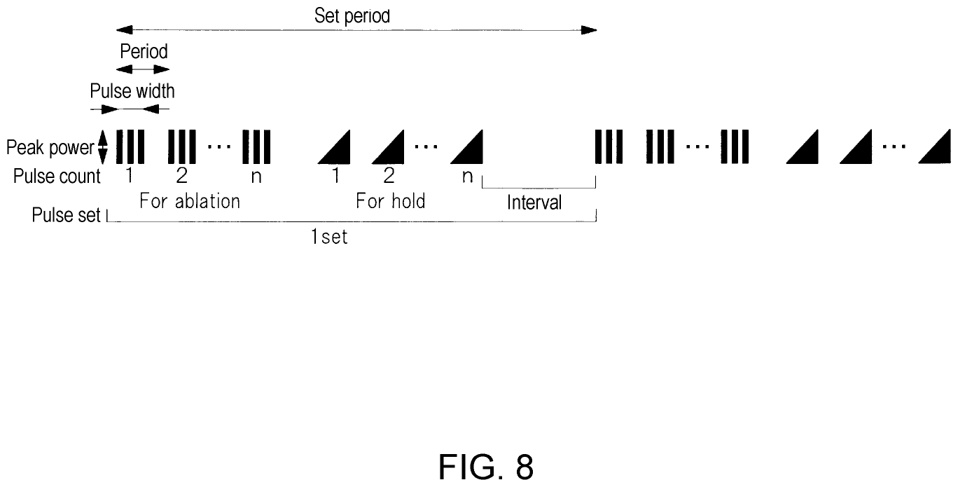

Specifically, the laser control device 5 switches the frequency of the laser light between, for example as shown in FIG. 8, a high-frequency first pulse group having an output for fragmenting the calculus S, and a second pulse group having a frequency lower than the first pulse group and having a waveform in which the peak power gradually increases in the axial direction of irradiation time.

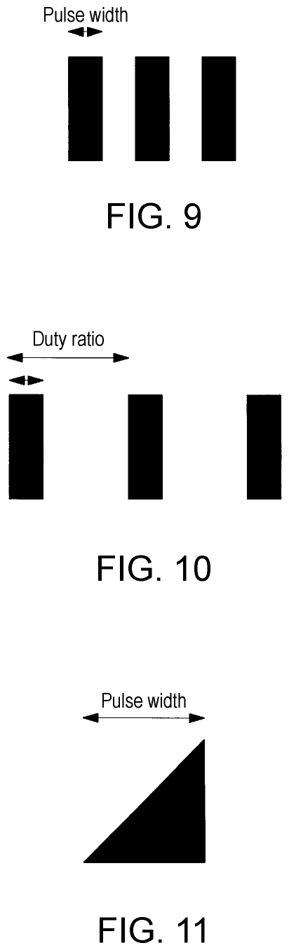

The waveform of the first pulse group is, for example as shown in FIG. 8 to FIG. 10, a multi-pulse train (MPT) in which the laser light is divided into several pulses and emitted. Set parameters of the first pulse group include the width of each pulse (pulse width) as shown in FIG. 9, or the duty ratio of the pulse width occupied across each pulse as shown in FIG. 10. The condition range of the pulse width of the first pulse group is, for example, 100 μs to 400 μs.

The waveform of the second pulse group is, for example as shown in FIG. 11, a triangular waveform in which the peak power gradually increases linearly in the axial direction of irradiation time. The condition range of the pulse width of the second pulse group is, for example, 400 μs to 1600 μs.

As shown in FIG. 8, the laser control device 5 repeatedly emits the first pulse group (for ablation) at predetermined cycles, and then switches the frequency to repeatedly emit the second pulse group (for hold) at predetermined cycles. After the emission of the second pulse group, an interval (stop period) is provided in which the emission of laser light is stopped for a certain period.

The laser control device 5 performs multiple pulse sets including the emission period of the first pulse group, the emission period of the second pulse group, and the interval. The above-described processes by the laser control device 5 are executed by the processor 15.

Next, the determination of each parameter at the time of determining the optimal control conditions by the determination part 9 will be described.

The determination part 9 determines each parameter that satisfies the following conditions: a total pulse energy PE within each pulse set is 0.5 J to 10 J, a frequency f of each pulse set is 0.5 Hz to 10 Hz, an average power Pₐᵥₑ of the laser light emitted in each pulse set is 1 W to 50 W per second, a frequency f_{A} of the first pulse group>a frequency f_{H} of the second pulse group, a pulse energy PE_{A} of the first pulse group within each pulse set>a pulse energy PE_{H} of the second pulse group within each pulse set.

The average power Pₐᵥₑ of the laser light emitted in each pulse set is calculated by the determination part 9 according to Equations (1), (2), and (3) as follows.

[Equation 1]

PE=PE_{A}·N_{A}+PE_{H}·N_{H} (1)

$$[ Equation 2 ]$$

$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} \qquad ( 2 )$$

$$[ Equation 3 ]$$

$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} \qquad ( 3 )$$

Here, Pₐᵥₑ represents the average power, PE_{A} represents the pulse energy of the first pulse group, PE_{H} represents the pulse energy of the second pulse group, f_{A} represents the frequency of the first pulse group, f_{H} represents the frequency of the second pulse group, N_{A} represents the pulse count of the first pulse group, N_{H} represents the pulse count of the second pulse group, and Tᵢₙₜ represents the interval.

The average power of the laser light is determined by the state of the calculus S or the state of the enclosed space where the laser light is irradiated. When the size or shape of the calculus S changes, the resistance between calculi S, the resistance at the wall of the enclosed space where the laser light is irradiated, or the resistance to the water flow changes, so the appropriate average power of the laser light also changes. When the number of the calculi S changes, the total weight of the calculi S also changes, so the appropriate average power of the laser light also changes. When the width of the enclosed space where the laser light is irradiated changes, the height at which the calculus S flutters also changes, so the appropriate average power of the laser light also changes. When the gravity direction in the enclosed space where the laser light is irradiated changes, the manner in which the calculus S remains around the fiber emission end 3a after laser light irradiation changes, so the appropriate average power of the laser light also changes.

In the present embodiment, the determination part 9 may determine the power of the first pulse group, the power of the second pulse group, and the length of the interval within a range where the average power Pₐᵥₑ of the laser light emitted in each pulse set becomes 20 W or less. Since the average power of the laser light in the pulse set decreases as the interval increases, by adjusting the length of the interval, the average power Pₐᵥₑ of the laser light can be kept at 20 W or less. The length of the interval is determined, for example, after the power of the first pulse group is determined and then the power of the second pulse group is determined.

Next, the relationship between the frequency as well as the fragmentation capability of the laser light and the suction effect will be described.

The first pulse group, which has a high frequency, has a shorter laser light irradiation interval than the second pulse group which has a low frequency. In the first pulse group, when the laser light is emitted from the fiber emission end 3a in liquid, bubbles are generated from the fiber emission end 3a, and the laser light is irradiated onto the calculus S by the laser light penetrating through the bubbles. Accordingly, although the calculus is fragmented, the calculus S moves in a direction away from the fiber emission end 3a due to the impact of fragmentation.

Subsequently, when the bubbles start to contract due to cooling by the surrounding liquid, a suction force is generated. As a result, within a certain range from the fiber emission end 3a, a suction effect acts to attract the calculus S present within the range toward the fiber emission end 3a. However, before the calculus S attracted to the fiber emission end 3a is positioned in the irradiation range of the laser light, the next laser light is emitted. Therefore, the laser light is either not irradiated onto the attracted calculus S or irradiated onto the edge of the calculus S. As a result, the calculus S rotates and moves again in a direction away from the fiber emission end 3a due to the impact of fragmentation.

Therefore, the first pulse group has high capability to fragment the calculus S by being emitted at high density in a short time, but tends to cause the calculus S to scatter easily.

Meanwhile, in the second pulse group which has a low frequency and a triangular waveform, when the laser light is emitted from the fiber emission end 3a in liquid, bubbles are generated from the fiber emission end 3a, and laser light is irradiated onto the calculus S by the laser light penetrating the bubbles. Then, the calculus S moves in a direction away from the fiber emission end 3a due to the impact of fragmentation.

Subsequently, when bubbles start to contract due to cooling by the surrounding liquid, a suction effect acts within a certain range from the fiber emission end 3a. Accordingly, the calculus S present within the certain range is attracted toward the fiber emission end 3a.

Subsequently, in a state where the calculus S attracted to the fiber emission end 3a is positioned in the irradiation range of the laser light, the next laser light is emitted. Accordingly, bubbles are generated again, and the laser light penetrating the bubbles is irradiated onto the calculus S. Then, due to the impact of fragmentation, the calculus S again moves in a direction away from the fiber emission end 3a. In this case as well, when the bubbles start to contract due to cooling by the surrounding liquid, a suction force is generated. As a result, a suction effect acts within a certain range from the fiber emission end 3a. Accordingly, the calculus S present within the certain range is attracted toward the fiber emission end 3a.

In other words, the second pulse group can cause the calculus S attracted by the suction effect to slip directly under the fiber emission end 3a. Therefore, although the second pulse group has low fragmentation capability, it has a high suction effect that attracts the calculus S toward the fiber emission end 3a within a certain range from the fiber emission end 3a. Particularly in the case of the second pulse group with a triangular waveform, after two or three bubbles are generated in a temporally adjacent state from the fiber emission end 3a, the bubbles connect to form snowman-shaped clustered bubbles. Accordingly, a higher suction effect than the laser light with a rectangular wave waveform can be achieved.

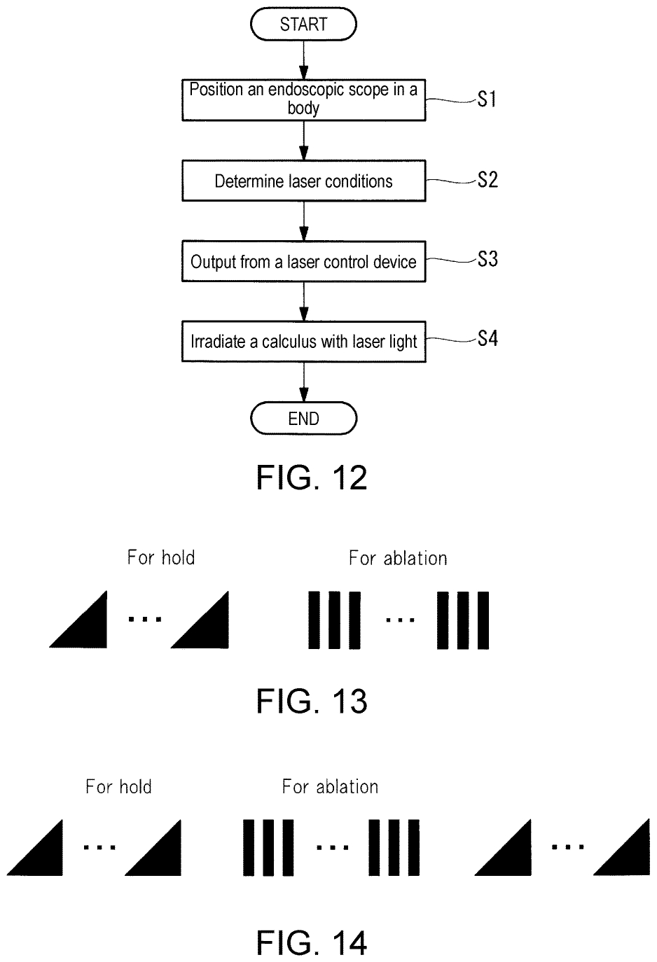

Next, the operation of the laser light irradiation system 1, the laser control device 5, and the laser light irradiation method having the above configuration will be described with reference to the flowchart of FIG. 12.

To fragment the calculus S present in the body by using the laser light irradiation system 1, the laser control device 5, and the laser light irradiation method according to the present embodiment, first, an endoscopic scope is positioned in the body (Step S1). Then, as shown in FIG. 2, after inserting the laser fiber 3 into the working channel 21a of the endoscopic scope 21, the fiber emission end 3a is positioned to face the calculus S as the treatment target.

Next, the user inputs the desired control conditions by using the manual input part 13, or selects the control conditions stored in the storage part 11, thereby setting the control conditions (laser conditions) for the frequency of the laser light (Step S2). The information regarding the state of the calculus S or the space may be acquired by the sensor 7 while fragmentating the calculus S, and the control conditions for the frequency of the laser light may be determined by the determination part 9 based on the acquired information. Then, the control conditions determined by the determination part 9 may be set.

Next, the laser control device 5 oscillates laser light from the light source unit based on the set control conditions (Step S3). Accordingly, the oscillated laser light is irradiated from the fiber emission end 3a onto the calculus S after passing through the laser fiber 3 (Step S4).

In Steps S3 and S4, as shown in FIG. 8, the laser control device 5 repeatedly emits laser light of the high-frequency first pulse group at the predetermined cycles, and then switches the frequency to repeatedly emit laser light of the second pulse group having a low frequency and a triangular waveform at the predetermined cycles. Thereafter, the emission of the laser light is stopped for a certain period. The pulse set including the emission periods and the interval are repeated a predetermined number of times.

In this case, when the high-frequency first pulse group is repeatedly emitted, the laser light emitted in a state where bubbles generated from the fiber emission end 3a are in contact with both the fiber emission end 3a and the calculus S is irradiated onto the calculus S by transmitting through the bubbles. Accordingly, the calculus S irradiated with the laser light is fragmented. Although the first pulse group has high fragmentation capability for fragmenting the calculus S, the calculus S is caused to scatter due to the impact of fragmentation. The scattered calculus S moves in a direction away from the fiber emission end 3a.

Next, by switching from the first pulse group to the second pulse group, the second pulse group with a low frequency and a triangular waveform is repeatedly emitted. In this case as well, the laser light that has transmitted through the bubbles generated from the fiber emission end 3a is irradiated onto the calculus S. While having low fragmentation capability for fragmenting the calculus S, the second pulse group has a high suction effect that acts when the bubbles start to disappear. Therefore, the calculus S present within a certain range from the fiber emission end 3a can be attracted to the fiber emission end 3a by the high suction effect and then slip directly under the fiber emission end 3a.

Therefore, within each pulse set, the calculus S that moves in a direction away from the fiber emission end 3a through the irradiation of the first pulse group can be retained at the fiber emission end 3a through the irradiation of the second pulse group. Then, in a state where the calculus S attracted to the fiber emission end 3a through the irradiation of the second pulse group is positioned within the irradiation range of the laser light, the high-frequency first pulse group of the next pulse set is emitted after the interval elapses. Accordingly, the laser light from the first pulse group is effectively irradiated onto the calculus S, allowing the calculus S to be fragmented with high efficiency.

Each pulse set is repeatedly implemented until the calculus S is fragmented to a desired size, such as less than 1 mm. In the case where the calculus S is fragmented to the desired size, there is no need to repeat the pulse sets, and it is sufficient to switch between the first pulse group and the second pulse group only once.

As described above, according to the laser light irradiation system 1, the laser control device 5, and the laser light irradiation method according to the present embodiment, by emitting the laser light of the first pulse group and the laser light of the second pulse group at different timings, the fragmentation effect exerted by the laser light of the first pulse group and the suction effect exerted by the laser light of the second pulse group can compensate for their respective disadvantages, and the fragmentation efficiency can be improved. Accordingly, wasteful firing of the laser light where the laser light is emitted in a state where the calculus S is positioned at a location away from the fiber emission end 3a can be suppressed, and the surgery time can be shortened.

Also, by providing an interval after the emission period in each pulse set, the average power of the laser light emitted in each pulse set can be suppressed. By suppressing the average power of the laser light in each pulse set to 20 W or less, the elevation of the patient's intrarenal temperature can be sufficiently suppressed.

In the present embodiment, in each pulse set, the second pulse group is emitted after the first pulse group is emitted, but the second pulse group may be emitted at least one of before and after the timing of emitting the first pulse group.

For example, as shown in FIG. 13, in each pulse set, the second pulse group may be emitted first, and then the first pulse group may be emitted by switching the frequency of the laser light. With this configuration, in each pulse set, the first pulse group can be irradiated in a state where the calculus S present within a certain range from the fiber emission end 3a is attracted to the fiber emission end 3a through irradiation of the second pulse group. In this case, an interval may be provided after emission of the first pulse group.

Also, for example, as shown in FIG. 14, in each pulse set, the second pulse group may be emitted both before and after the first pulse group is emitted. With this configuration, in each pulse set, the first pulse group can be irradiated in a state where the calculus S present within a certain range from the fiber emission end 3a is attracted to the fiber emission end 3a through the irradiation of the second pulse group, and the calculus S that moves in a direction away from the fiber emission end 3a through the irradiation of the first pulse group can be retained at the fiber emission end 3a through the irradiation of the second pulse group. In this case, an interval may be provided after emitting in the order of the second pulse group, the first pulse group, and the second pulse group.

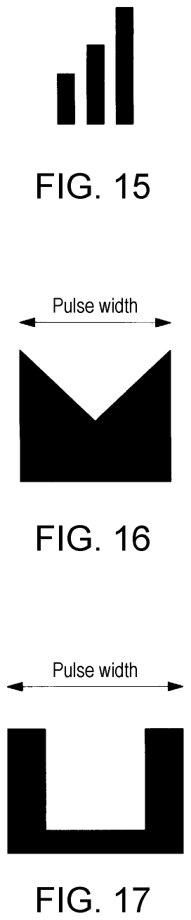

Also, in the present embodiment, a triangular waveform that increases linearly is exemplified and described as the waveform of the second pulse group, but any waveform in which the peak power gradually increases in the axial direction of irradiation time may be used, and the waveform is not limited to a triangular waveform. For example, as shown in FIG. 15, a waveform that increases stepwise in the axial direction of irradiation time may be used, or a waveform that increases gradually in a smooth curved manner may be used.

Also, the waveform of the second pulse group may be an M-shaped waveform in which the peak power gradually decreases and then gradually increases in the axial direction of irradiation time as shown in FIG. 16, or may be a U-shaped waveform in which the peak power sharply decreases once from a high state in the axial direction of the irradiation time, continues in the decreased state for a certain time, and then sharply increases again as shown in FIG. 17. With any of the waveforms, the condition range of the pulse width for the second pulse groups may be 400 μs to 1600 μs.

In the M-shaped waveform and the U-shaped waveform, the peak power at the center in the axial direction of irradiation time may be within 50% of the peak power at both ends in the axial direction. By arranging the maximum peaks separately at both ends in the axial direction with the peak power within 50% of the center in the axial direction in between, two bubbles are generated separately from each other from the fiber emission end 3a. Accordingly, a high suction effect is attained.

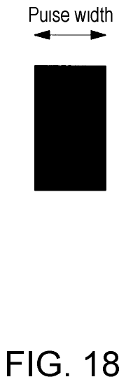

A rectangular wave waveform as shown in FIG. 18 may be adopted as the second pulse group. Even in the case where a rectangular wave is adopted, the suction effect itself can be obtained, but the lump bubbles formed in the case of a triangular waveform and the two separate bubbles generated in the case of an M-shaped waveform and a U-shaped waveform are not generated with the rectangular wave.

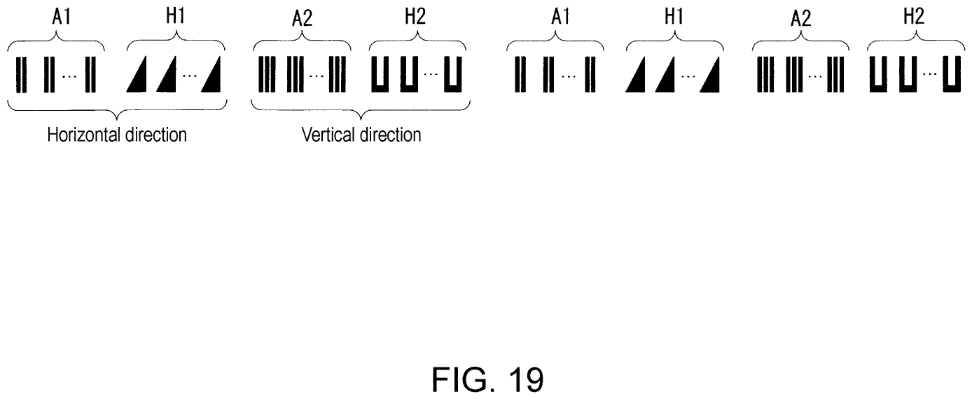

In the present embodiment, for example, as shown in FIG. 19, the pulse count of the first pulse group may be varied per pulse set, and the waveform of the second pulse group may be varied per pulse set.

In the example shown in FIG. 19, a first pulse set including a high-frequency first pulse group A1 of a multi-pulse train with a reduced number of divided pulses, a second pulse group H1 having a frequency lower than the first pulse group A1 and having a triangular waveform, and an interval, and a second pulse set including a high-frequency first pulse group (third pulse group) A2 of a multi-pulse train with an increased number of divided pulses compared to the first pulse group A1, a second pulse group (fourth pulse group) H2 having a frequency lower than the first pulse group A2 and having a U-shaped waveform, and an interval are alternately repeated.

The first pulse group A2 may have a high frequency different from the first pulse group A1, and may be, for example, one with a reduced number of divided pulses compared to the first pulse group A1. Also, the second pulse group H2 may have a waveform having a frequency different from the second pulse group H1 and lower than the first pulse group A2, and have peak power gradually increasing in the axial direction of irradiation time.

Here, the approach direction of the laser fiber 3 to the calculus S in the renal calyx C differs for each renal calyx C where the calculus S remains.

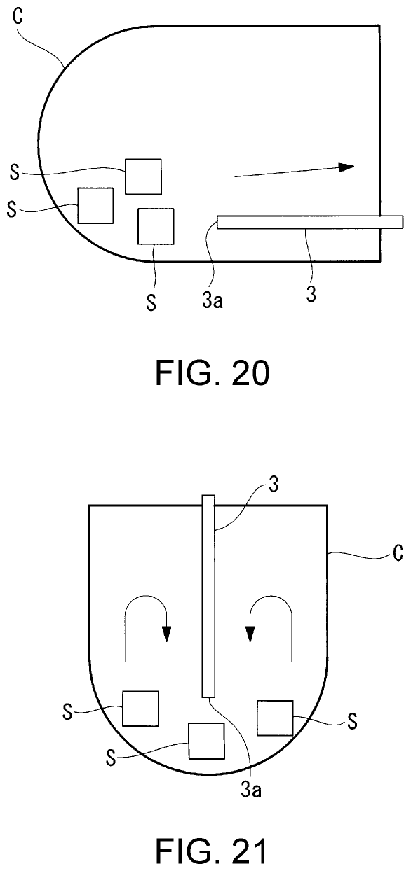

In the renal calyx C where the approach direction faces the horizontal direction, for example, as shown in FIG. 20, when the calculus S scatters from the fiber tip 3a toward the base end side of the laser fiber 3 due to the impact of laser light, due to gravity, the calculus S does not return to the irradiation position of the laser light. Therefore, in this case, control conditions that emphasize the suction effect are desirable.

On the other hand, in the renal calyx C where the approach direction faces the gravity direction, for example, as shown in FIG. 21, when the calculus S scatters from the fiber tip 3a toward the base end side of the laser fiber 3 due to the impact of the laser light, the calculus S returns to the irradiation position of the laser light due to gravity. Therefore, in this case, control conditions that emphasize fragmentation are desirable.

Also, in the case where the calculus S is positioned in the irradiation range of the laser light at the time of irradiation of the second pulse group, the calculus S may be fragmented by the laser light of the second pulse group.

The second pulse groups with the triangular waveform and the M-shaped waveform have a suction effect during the period in which the peak power is low, but the average power is reduced, so it is difficult to scatter the calculus S. Therefore, the second pulse groups with the triangular waveform and the M-shaped waveform are suitable for the renal calyx C where the approach direction faces the horizontal direction and scattering of the calculus S is undesirable as much as possible, and can enhance the fragmentation efficiency in such renal calyx C.

On the other hand, the second pulse groups with the U-shaped waveform and the rectangular wave waveform have a high average power per pulse due to a long duration of the maximum peak power, and therefore exhibits not only the suction effect but also high fragmentation capability, though such second pulse groups tend to scatter the calculus S during fragmentation. Therefore, the second pulse groups with the U-shaped waveform and the rectangular wave waveform are suitable for the renal calyx C where the approach direction faces the gravity direction and the calculus S returns even after scattering, and can enhance the fragmentation efficiency in such renal calyx C.

In the example shown in FIG. 19, the first pulse set having the high-frequency first pulse group A1 with a reduced number of divided pulses and the low-frequency second pulse group H1 with a triangular waveform is particularly effective in the renal calyx C where the approach direction is horizontal. On the other hand, the second pulse set having the high-frequency first pulse group A2 with an increased number of divided pulses and the low-frequency second pulse group H2 with a U-shaped waveform is particularly effective in the renal calyx C where the approach direction is the gravity direction.

As shown in FIG. 19, by appropriately combining the first pulse set having the second pulse group with a triangular waveform or an M-shaped waveform and the second pulse set having the second pulse group with a U-shaped waveform or a rectangular wave waveform, it can be applied to the renal calyx C where the approach direction is both the horizontal direction and the gravity direction, and the calculus S can be comprehensively fragmented. Also, it is effective in the renal calyx C where the approach direction is inclined at an angle between the horizontal direction and the gravity direction, and is also effective in the case of moving back and forth between several renal calyces C with different orientations.

Also, in the present embodiment, the frequency of the laser light is switched between the first pulse group and the second pulse group. Instead of this, the laser control device 5 may switch the frequency of the laser light to three or more different frequencies.

Also, in the present embodiment, the determination part 9 is provided separately from the processor 15 of the laser control device 5, but instead of this, the processor 15 of the laser control device 5 may also serve the function of the determination part 9.

Also, as the peak power increases, the bubbles generated from the fiber emission end 3a also increase. Also, as the pulse width of the laser light increases, the number of bubbles that are repeatedly generated and disappear in one pulse increases. Since the phenomena affect the effect of fragmenting the calculus S and the suction effect, by executing a process to optimize the laser conditions in the determination part 9, not only the frequency but also the peak power, the pulse width, the irrigation flow rate, etc., may be changed. As the type of the calculus S differs, the hardness and the density of the calculus S differ due to differences in composition, so how the calculus S is fragmented also changes. Therefore, sensing the type of the calculus S is useful for optimizing the frequency, the peak power, and the pulse width.

Also, in the present embodiment, the control conditions by the determination part 9 can be adopted for controlling the frequency of the laser light. Instead of this, it may also be that the laser light irradiation system 1 does not include the sensor 7 and the determination part 9 and adopts the control conditions input by the user through the manual input part 13 and the control conditions stored in the storage part 11. In this case, Step S2 to Step S4 may be executed based on each control condition. In the case of adopting the control conditions stored in the storage part 11, the parameters of the first pulse group and the second pulse group can be set more quickly and simply.

Also, in the present embodiment, the determination part 9 may determine whether the calculus S has been fragmented until it becomes smaller than a predetermined threshold, based on the information regarding the state of the calculus S. In the case of determining that the calculus S is not fragmented until it becomes smaller than the predetermined threshold, Step S2 to S4 may be repeated. Also, it may also be that, in the case of determining that the calculus S has been fragmented until it becomes smaller than the predetermined threshold, an instruction to end laser irradiation is notified from the determination part 9 to the laser control device 5.

The present embodiment describes urinary calculi as an example, but the effect of multi-pulse train is not limited to urinary calculus treatment and can be similarly obtained for bile duct calculi. Also, the present example is not limited to urinary calculi and can be applied to the treatment of any biological tissue in water, in an aqueous solution, in an organic solution, or in an atmosphere.

As described above, the embodiments of the disclosure have been described in detail with reference to the drawings, but the specific configuration is not limited to these embodiments, and design changes within the scope that do not depart from the gist of the disclosure are also included. For example, the disclosure is not limited to applications to the above embodiments and modification examples, and may be applied to embodiments that appropriately combine these embodiments and modification examples, and is not particularly limited.

What is claimed is:

1. A laser light irradiation system that fragments a calculus in liquid by irradiating laser light to the calculus present in a body, the laser light irradiation system comprising:
a laser fiber, having a fiber emission end that emits the laser light; and
at least one processor, configured to control a frequency of the laser light emitted from the fiber emission end,
wherein the processor is configured to switch the frequency of the laser light between a high-frequency first pulse group having an output that fragments the calculus and a second pulse group having a frequency lower than the first pulse group and having a waveform in which a peak power gradually increases in an axial direction of irradiation time, and emit the second pulse group at least one of before and after a timing of emitting the first pulse group.

2. The laser light irradiation system as claimed in claim 1, wherein a waveform of the first pulse group is a multi-pulse train.

3. The laser light irradiation system as claimed in claim 1, wherein the waveform of the second pulse group is a triangular waveform in which a peak power gradually increases in the axial direction, or an M-shaped waveform in which a peak power gradually decreases and then gradually increases in the axial direction.

4. The laser light irradiation system as claimed in claim 1, wherein the processor is configured to perform a pulse set a plurality of times, the pulse set providing a stop period for stopping emission of the laser light for a predetermined period after an emission period comprising a period of emission of the first pulse group and a period of emission of the second pulse group.

5. The laser light irradiation system as claimed in claim 4, wherein the processor is configured to determine a power of the first pulse group, a power of the second pulse group, and a length of the stop period within a range where an average power of the laser light emitted in the pulse set is 20 W or less.

6. The laser light irradiation system as claimed in claim 5, wherein the average power is calculated by Equations (1), (2), and (3) as follows:
[Equation 1]
PE=PE_{A}·N_{A}+PE_{H}·N_{H} (1)
$$[ Equation 2 ]$$
$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} , and \qquad ( 2 )$$
$$[ Equation 3 ]$$
$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} , \qquad ( 3 )$$
wherein Pₐᵥₑ represents the average power, PE_{A} and PE_{H} represent pulse energies, f_{A} and f_{H} represent frequencies, N_{A} and N_{H} represent pulse counts, and Tᵢₙₜ represents the stop period.

7. The laser light irradiation system as claimed in claim 4, wherein the processor is configured to perform a second pulse set in which, following the first pulse set, an emission period is followed by the stop period, the emission period comprising: a period of emission of a third pulse group having an output for fragmenting the calculus and having a high frequency different from the first pulse group; and a period of emission of a fourth pulse group having a frequency different from the second pulse group and lower than the third pulse group and having a waveform in which a peak power gradually increases in the axial direction of irradiation time.

8. The laser light irradiation system as claimed in claim 1, comprising:
an endoscope configured to be inserted into the body,
wherein the endoscope comprises a channel that allows the fiber emission end to protrude forward in a state where the laser fiber penetrates.

9. A laser control device, comprising:
at least one processor, configured to control a frequency of laser light emitted from a fiber emission end of a laser fiber toward a calculus present in a body,
wherein the processor is configured to switch a frequency of the laser light between a high-frequency first pulse group having an output that fragments the calculus and a second pulse group having a frequency lower than the first pulse group and having a waveform in which a peak power gradually increases in an axial direction of irradiation time, and emit the second pulse group at least one of before and after a timing of emitting the first pulse group.

10. The laser control device as claimed in claim 9, wherein a waveform of the first pulse group is a multi-pulse train.

11. The laser control device as claimed in claim 9, wherein the waveform of the second pulse group is a triangular waveform in which a peak power gradually increases in the axial direction, or an M-shaped waveform in which a peak power gradually decreases and then gradually increases in the axial direction.

12. The laser control device as claimed in claim 9, wherein the processor is configured to perform a pulse set a plurality of times, the pulse set providing a stop period for stopping emission of the laser light for a predetermined period after an emission period comprising a period of emission of the first pulse group and a period of emission of the second pulse group.

13. The laser control device as claimed in claim 12, wherein the processor is configured to determine a power of the first pulse group, a power of the second pulse group, and a length of the stop period within a range where an average power of the laser light emitted in the pulse set is 20 W or less.

14. The laser control device as claimed in claim 13, wherein the average power is calculated by Equations (1), (2), and (3) as follows:
[Equation 1]
PE=PE_{A}·N_{A}+PE_{H}·N_{H} (1)
$$[ Equation 2 ]$$
$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} , and \qquad ( 2 )$$
$$[ Equation 3 ]$$
$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} , \qquad ( 3 )$$
wherein Pₐᵥₑ represents the average power, PE_{A} and PE_{H} represent pulse energies, f_{A} and f_{H} represent frequencies, N_{A} and N_{H} represent pulse counts, and Tᵢₙₜ represents the stop period.

15. The laser control device as claimed in claim 12, wherein the processor is configured to perform a second pulse set in which, following the first pulse set, an emission period is followed by the stop period, the emission period comprising: a period of emission of a third pulse group having an output for fragmenting the calculus and having a high frequency different from the first pulse group; and a period of emission of a fourth pulse group having a frequency different from the second pulse group and lower than the third pulse group and having a waveform in which a peak power gradually increases in the axial direction of irradiation time.

16. A laser light irradiation method for fragmenting a calculus in liquid by irradiating laser light to the calculus present in a body, the laser light irradiation method comprising:
emitting a high-frequency first pulse group having an output for fragmenting the calculus from a fiber emission end toward the calculus; and
at least one of before and after a timing of emitting the first pulse group, emitting a second pulse group having a frequency lower than the first pulse group and having a waveform in which a peak power gradually increases in an axial direction of irradiation time.

17. The laser light irradiation method as claimed in claim 16, comprising:
performing a pulse set a plurality of times, the pulse set providing a stop period for stopping emission of the laser light for a predetermined period after an emission period comprising a period of emission of the first pulse group and a period of emission of the second pulse group.

18. The laser light irradiation method as claimed in claim 17, comprising:
determining a power of the first pulse group, a power of the second pulse group, and a length of the stop period within a range where an average power of the laser light emitted in the pulse set is 20 W or less.

19. The laser light irradiation method as claimed in claim 18, wherein the average power is calculated by Equations (1), (2), and (3) as follows:
[Equation 1]
PE=PE_{A}·N_{A}+PE_{H}·N_{H} (1)
$$[ Equation 2 ]$$
$$f = \frac{{f}_{A} {f}_{H}}{{f}_{H} \cdot {N}_{A} + {f}_{A} \cdot {N}_{H} + {T}_{i n t} \cdot {f}_{A} \cdot {f}_{H}} , and \qquad ( 2 )$$
$$[ Equation 3 ]$$
$${P}_{ave} = PE \cdot f = \frac{{PE}_{A} \cdot {N}_{A} + {PE}_{H} \cdot {N}_{H}}{{f}_{H} \cdot {N}_{A} + {f}_{A} \cdot {N}_{H} + {T}_{i n t} \cdot {f}_{A} \cdot {f}_{H}} {f}_{A} \cdot {f}_{H} , \qquad ( 3 )$$
wherein Pₐᵥₑ represents the average power, PE_{A} and PE_{H} represent pulse energies, f_{A} and f_{H} represent frequencies, N_{A} and N_{H} represent pulse counts, and Tᵢₙₜ represents the stop period.

20. The laser light irradiation method as claimed in claim 17, comprising:
performing a second pulse set in which, following the first pulse set, an emission period is followed by the stop period, the emission period comprising: a period of emission of a third pulse group having an output for fragmenting the calculus and having a high frequency different from the first pulse group; and a period of emission of a fourth pulse group having a frequency different from the second pulse group and lower than the third pulse group and having a waveform in which a peak power gradually increases in the axial direction of irradiation time.
